(19) United States
(12) Patent Application Publication
(10) Pub. No.: US 2008/0280934 A1
(43) Pub. Date: Nov. 13, 2008

(54) Method of Preparing Porphyrin Derivatives, Porphyrin Derivatives, Uses Thereof and Pharmaceutical Compositions

(76) Inventors: Johannis Lugtenburg, Oegstgeest (NL); Hendrikus Nicolaas Sebastiaan Van der Haas, Leiden (NL); Johannes Joseph Schuitmaker, Leiden (NL); Robertus Leendert Pieter De Jong, Sassenheim (NL)

Correspondence Address: MAYER BROWN LLP, P.O. BOX 2828, CHICAGO, IL, 60690, US

(21) Appl. No.: 10/583,082

(22) PCT Filed: Dec. 20, 2004
(86) PCT No.: PCT/NL04/00888

§ 371 Date: Aug. 8, 2007

(30) Foreign Application Priority Data
Dec. 18, 2003 (NL) 1025049

(51) Int. Cl. A61K 31/4745; C07D 471/22; A61P 17/00; A61P 35/00; A01N 43/90; A61P 15/00; A61P 37/00; A61P 27/02

(52) U.S. Cl. 514/279; 540/145

(57) ABSTRACT

Method of preparing a porphyrin derivative starting from a meso-substituted porphyrin compound. According to the invention, the meso-substituted porphyrin compound used is a meso-(2′-cyanovinyl)-substituted porphyrin compound, wherein said meso-(2′-cyanovinyl)-substituted porphyrin compound, in a form in which its porphyrin ring is complexed with a bivalent metal ion is converted, in the present of an acid for which 0<pKa<5 and an oxidizing agent; or in the presence of a Vilsmeier reagent, into a porphyrin derivative having a quinoline-ring system ring peri-condensed to the porphyrin ring.

The present invention relates to a method of preparing porphyrin derivatives starting from a meso-substituted porphyrin compound.

Such a method is known in the art. In particular Arnold, D. et al. J. Chem. Soc. Perkin 1, pp. 1660-1670 (1978) disclose the formation of benzochlorins starting from a meso-substituted acrylalcohol- or acrylaldehydeporphyrin compound.

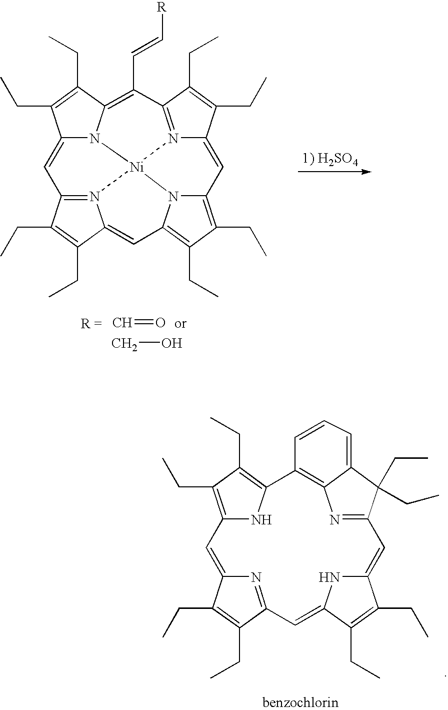

An important aspect is that such compounds can be prepared starting from hemin and protoporphyrin, which are relatively inexpensive and commercially available in relatively large amounts. This does not apply for many other porphyrins described in the literature and restricts the application thereof significantly. The method disclosed by Arnold is useful for the preparation of modified porphyrin compounds having an absorption maximum (λmax) that is shifted towards the red relative to the starting porphyrin.

For many applications of porphyrins it is important to have at one's disposal porphyrin derivatives having a λmax in a particular wavelength range. An example thereof are photo-sensitizers which are used for photodynamic therapy or for the disinfection of blood products. Hence, it is important to have at one's disposal a wide range of chemical reactions for the synthesis of such porphyrin compounds starting from another porphyrin compound, such as, but not limited to, hemin and protoporphyrin.

The object of the present invention is to provide a new method for the preparation of porphyrin derivatives having a modified λmax. The object of the present invention is to also provide a method for the preparation of porphyrin derivatives possessing an increased hydrophilic nature, wherein said porphyrin derivatives consequently may be more suitable for pharmaceutical applications.

To this end the method according to the invention is characterized in that a meso-(2′-cyanovinyl)-substituted porphyrin compound of which the vinyl is optionally substituted is used as the meso-substituted porphyrin compound, wherein said meso-(2′-cyanovinyl)-substituted porphyrin compound, in a form in which its porphyrin macrocycle is complexed with a bivalent metal ion

i) is subjected to
an acid for which 0<pKa<5
and
an oxidizing agent,

with the restriction that if the carbon atom of the porphyrin macrocycle at which the (2′-cyanovinyl) substituent is attached is designated Cα, there must be a substituent attached to Cδ, counting along the perimeter of the porphyrin macrocycle, said substituent comprising a —C—C motif directly attached at the Cδ carbon atom; or

ii) is subjected under aprotic conditions to a Vilsmeier reagent having a reactive motif

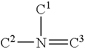

containing a quaternary nitrogen atom which is directly linked to two carbon atoms C¹, C² wherein said carbon atoms are not part of a unsaturated or aromatic moiety, and which quaternary nitrogen atom is directly linked to a carbon atom C³ via a double bond, said carbon atom C³ carrying a halogen atom chosen from fluoro, chloro, bromo and iodo with the restriction that if the carbon atom of the porphyrin macrocycle at which the (2′-cyanovinyl) substituent is attached is designated Cα, there must be a substituent attached to Cδ, counting along the perimeter of the porphyrin macrocycle, said substituent comprising a —CH motif directly attached at the Cδ carbon atom;

to convert said meso-(2′-cyanovinyl)-substituted porphyrin compound into a porphyrin derivative having a quinoline-ring system peri-condensed to the porphyrin ring, and optionally the bivalent metal ion is removed or replaced by another metal ion, and optionally the nitrogen atom of the quinoline-ring system ring is quaternized.

Surprisingly, this method results in the formation of porphyrin derivatives having a ring system with two aromatic rings fused to the porphyrin macrocycle, in particular peri-annulated quinoporphyrins, which are entirely new compounds. These new compounds possess an aromatic nitrogen atom which contributes to the amfiphilic nature of the new compounds compared to porphyrin compounds known in the art. This helps to increase solubility in polar solvents, such as aqueous solutions, and in particular in bodily fluids such as blood.

The oxidizing agent in the alternative reaction step i) is conveniently oxygen, for example from the air. The particular acid to be used depends on the particular substituents of the vinyl group of meso-(2′-cyanovinyl), and a suitable acid can be found without undue effort using routine experimentation by starting with acids having different pKa's in the range of 0 to 5. Strong acids (pKa<0) are to be avoided before the reaction is complete because the yield will decrease due to demetallation of the porhyrin macrocycle and hydrolysation of the nitril of the meso-(2′-cyanovinyl) group. For the sake of convenience, in the present description the term meso-(2′-cyanovinyl) also includes derivatives of this group where vinyl carries a substituent on one or both carbon atoms of vinyl, even if this has not been mentioned explicitly or if the term is part of a structural formula, which has been done for the purpose of readability only. The Vilsmeier reagent of alternative step ii) is conveniently prepared in situ starting from a carbonyl amide, for example using POX₃ where X is a halogen atom, such as chlorine. Other reactions for preparing Vilsmeier reagents are known in the art and do not form part of the invention. The reaction with the Vilsmeier reagent may be performed at room temperature, whereas the reaction according to step i) will generally be performed at an elevated temperature.

The method according to the present invention was shown to be very suitable for the preparation of porphyrin derivatives wherein for alternative step i) a meso-(2′-cyanovinyl)-substituted porphyrin compound of formula (I) is used as the starting compound,

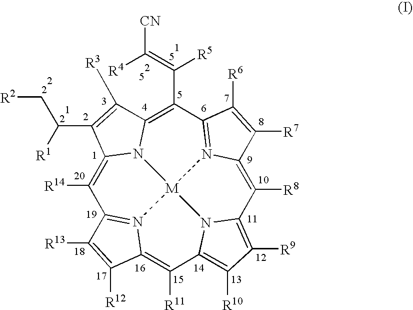

or wherein for alternative step ii) meso-(2′-cyanovinyl)-substituted porphyrin compound of formula (III) is used as the starting compound:

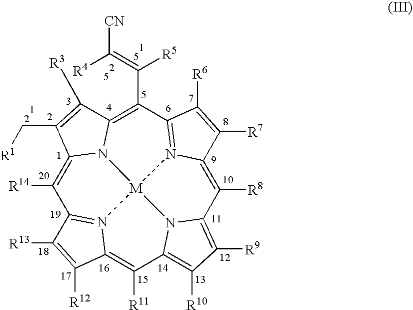

wherein

R¹; R² represent independently of each other hydrogen, linear or branched (C₁₋₈) alkyl, or linear or branched (C₁₋₈) alkyl C(O)(O) (C₁₋₈alkyl, wherein the groups comprising alkyl may optionally be substituted with fluoro, chloro, bromo, iodo, nitrile, (C₁₋₈) thioether, and (C_{1-8) alkoxy;}
R² represents H or (C₁₋₈) alkyl;
R⁴ and R⁵, represent, independently of each other, hydrogen, nitrile, monocyclic, bicyclic or tricyclic (C₆-₁₄) aryl, or (C₁-₄) alkyl wherein the aryl and alkyl group may optionally be substituted with fluoro, chloro, bromo, iodo, nitrile, (C₁₋₈) thioether, and (C₁-C₈) alkoxy;

R⁶ to R¹⁴ represent independently of each other, hydrogen, linear or branched (C₁₋₈) alkyl, linear or branched (C₁₋₈) alkyl C(O)O (C₁₋₈)alkyl, wherein n is an integer of 0 to 4, CH₂═CH—, a monocyclic, bicyclic or tricyclic (C₃-C₁₄) aryl, which aryl may optionally contain one or more nitrogen atoms as heteroatoms; and R⁸, R¹¹, and R¹⁴ may in addition represent an acrylonitrile group substituted with R⁴′ and R⁵′, wherein R⁴′ and R⁵′ are as defined for R⁴ and R⁵; and

M represents a bivalent metal ion, wherein the compound of formula (I) or (III) is converted into the corresponding porphyrin derivative of formula (II) comprising a quinoline-ring system fused to the porphyrin ring

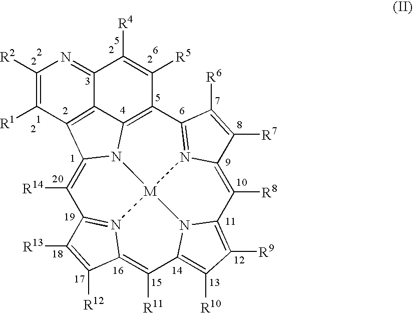

wherein the substituents have the meanings given above, and depending on the meaning of R⁸, R¹¹, and R¹⁴ and the correspondence of an adjacent R⁷, R⁹, R¹⁰, R¹², and R¹³ with R³, optionally more than one quinoline-ring system peri-condensed to the porphyrin ring is present.

In the method according to the invention the meso-(2′-cyanovinyl)-substituted porphyrin compound of formula (I) is preferably prepared by introducing a formyl or acetyl residue at a meso position of a porphyrin compound, after which the thus formed mesoformylporphyrin or mesoacetyl porphyrin is converted into its meso-(2′-cyanovinyl) derivative or meso (2′ -cyano-1′ -methylvinyl) derivative, respectively. According to a preferred embodiment, the mesoformylporphyrin formed is converted into the meso-(2′-cyanovinyl)-substituted porphyrin compound of formula (I) by reaction with diethylphosphonoace-tonitrile. Other reactions are easily available in accordance with the invention, for example with malononitrile (NCCH₂CN) to result in a meso-(2′,2′-dicyanovinyl)-substituted porphyrin compound where R⁴ is nitrile. Thus, the starting material, the meso-(2′-cyanovinyl) compound of formula (I), for performing the method according to the invention is obtained in a simple manner. Usually the porphyrin compound used as a starting material will contain a hydrogen atom at at least one meso position (i.e., at least one of R⁸, R¹¹ and R¹⁴ is hydrogen).

In case a substituent R⁴ or R⁵ is an aryl group, the aryl group is usually phenyl, naphthyl, phenanthryl or anthracyl. The ester groups, such as those of R¹⁰ and R¹², may optionally be hydrolysed to yield carboxylic acids, further improving the solubility of the present porphyrin derivatives in aqueous solutions. These groups also provide excellent starting points to couple the porphyrin derivatives to other molecules, such as proteins, or substrates using methods for coupling well known in the art. Also, is it possible to attach one or more groups to the porphyrin derivative, for example to one or more carboxylic acid groups obtained as described above. For example, improve solubility in aqueous solutions a group comprising a PEG tail may be introduced in the porphyrin derivative. Alternatively, more charged groups may be introduced.

According to a very favourable embodiment, the nitrogen atom of-the peri-condensed quinoline-ring system in formula (I) is quaternized.

This results in porphyrine compounds that have an improved solubility in water thanks to the positive charge on the quaternary nitrogen atom(s), as a result of which the solubility is less dependent on pH. Even more interesting, such porphyrin derivatives have absorption maxima in the far red (say 750 nm). The group substituting the nitrogen atom(s) is preferably alkyl and aryl as defined for R6 but may also be, for example, alkaryl such as benzyl and optionally substituted as for R6. The particular choice will depend on the desired nature of the porphyrin derivative.

An advantageous embodiment is characterized in that the porphyrin compound used as a starting material for the preparation of the meso-(2′-cyanovinyl) porphyrin is chosen from the group of i) hemin, and ii) heme.

The synthesis of porphyrin derivatives, in particular on a large scale (hundred grams or more) and with high purity has been a problem until now. The simple chemical reaction method according to the invention makes it possible to obtain porphyrin derivatives with excellent yield and good purity after limited purification.

To perform a reaction according to the invention it is preferred that Ni²⁺ is used as the bivalent metal ion.

This was shown to result in good yields. If desired, the metal ion may be removed (by introducing two hydrogen atoms) or replaced after reaction, using methods well known in the art (References: Fuhrop, J. H. et al in Porphyrins and Metalloporphyrins, Smith, K. M.; Ed Elsevier: Amsterdam, 1975; p. 185 and pp. 795-798. Buchler, J. W.; In The Porphyrins; Dolphin, D.; Ed.; Academic-Press, New York 1978, Vol 1A, p. 389. Sanders et al.., In The Porphyrin Handbook, Kadish, K. M., Smith, K. M. and Guilard, R.;, Ed.; Academic Press, San Diego, 2000,Vol 3, chapter 15, pp.3-40. demetallation of porphyrins: Fuhrop, J. H. et al in Porphyrins and Metalloporphyrins, Smith, K. M.; Ed Elsevier: Amsterdam, 1975; pp. 195-207 and pp. 243-247. Particular reference is made to Buchler, J. W. et al, Liebigs annalen der Chemie (1988), pp. 43-54).

To achieve good yields and purities, it is preferred to perform the reaction according to the invention with a Brön-sted-acid, using a Bronsted-acid with the provisio that 0<pKa<5, at a temperature above 140° C.

Lower temperatures, for example 90° C. may also be employed successfully, depending on the particular nature of the substituents involved in the reaction. In general, pKa values between 0.5 and 2.5 are preferred, but again this will depend on the particular nature of the substituents involved in the reaction. It is well within the skill of an ordinary person skilled in the art to determine suitable reaction conditions using routine experimentation using the disclosed examples as guidance.

Preferably the Vilsmeier reagent used is of formula (IV)

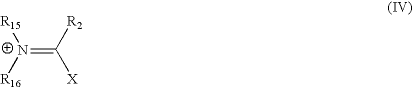

wherein

R15 and R16 are, independently of each other, linear or branched C₁₋₈ alkyl,

X is fluoro, chloro, bromo and iodo, and

R2 is hydrogen, linear or branched (C₁₋₈)alkyl, or linear or branched (C₁₋₈)alkyl C(O)O (C₁₋₈)alkyl, wherein the groups comprising alkyl may optionally be substituted with fluoro, chloro, bromo, iodo, nitrile, (C₁-₈) thioether, and (C₁₋₈)-alkoxy.

Such Vilsmeier reagents are sufficiently electrophilically hard obtain the desired quinoporphyrins. It is preferred that X is chloro or bromo.

In addition the invention relates to every porphyrin derivative having a quinoline-ring system peri-condensed to the porphyrin ring, and more specifically a porphyrin derivative having a quinoline-ring system peri-condensed to the porphyrin ring obtainable with the method according to the invention. Please note that this includes derivatives having more than one quinoline-ring system.

More specifically the invention relates to porphyrin derivatives, wherein said derivatives are:
2′-methoxycarbonylquino[4, 4a,5, 6-jkl]-annulated 12-demethyl-13-de[2-(methoxycarbonyl)ethyl]mesoporphyrin dimethylester;
2′-methoxycarbonylquino[4, 4a,5, 6-qrs]-annulated 18-demethyl-17-de[2-(methoxycarbonyl)ethyl]mesoporphyrin dimethylester;
quino[4,4a,5, 6-abt]-annulated 2-demethyl-3-deethylmesoporphyrin dimethylester;
quino[4,4a,5, 6-efg]-annulated 7-demethyl-8-deethylmesoporphyrin;
2′-methoxycarbonylquino[4,4a,5,6-jkl]-annulated 12-demethyl-13-de[2-(methoxycarbonyl)ethyl]mesoporphyrin;
2′-methoxycarbonylquino[4,4a,5,6-qrs]-annulated 18-demethyl-17-de [2-(methoxycarbonyl) ethyl]mesoporphyrin;
quino[4,4a,5,6-abt]-annulated 2-demethyl-3-deethylmesoporphyrin;
quino[4,4a,5,6-bcd]-2-demethyl-3-deethylmesoporphyrin dimethylester;
quino[4,4a, 5,6-bcd]-2-demethyl-3-deethylmesoporphyrin;
3′-methylquino[4,4a,5,6-efg]-7-demethyl-8-deethylmesoporphyrin dimethylester;
3′-methylquino[4,4a,5, 6-efg]-7-demethyl-8-deethylmesoporphyrin;
9′-aminocarbonylquino[4,4a,5,6-efg]-7-demethyl-8-deethylquinoporphyrin dimethylester;
9′-amindcarbonylquino[4,4a,5,6-efg]-7-demethyl-8-deethylquinoporphyrin benzylquinolinium[4,4a,5,6-efg]-annulated mesoporphyrin dimethylester
N-benzylquinolinium[4,4a,5,6-efg]-annulated mesoporphyrin.

The invention also contemplates an (optionally substituted) meso-(2′-cyanovinyl)porphyrin useful as a starting material for the preparation of a corresponding quinoline-ring systemporphyrin derivative. Optionally substituted refers to both vinyl and the porphyrin macro-cycle.

Finally, the invention relates to the use of a porphyrin derivative according to the invention for the preparation of a pharmaceutical composition for treating those indications that are well known in the art for clinical and other uses of photosensitizers. These indications include amongst others:

1) Skin and mucosa disorders: benign, malignant, inflamed and infectious skin/mucosa disorders such as acne, warts, eczema, birthmarks (including vascular malformations such as naevus flammeus and hyperpigmentation), hirsutism, skin/(burn)wound/mucosa infections (caused by bacteria, viruses, dermatophytes and other fungi, yeasts and/or parasites), actinic keratoses, psoriasis, primary tumors (including basal cell carcinomas, squamous cell carcinoma and melanomas) and secondary tumors of the skin and mucosa. In addition, photosensitizers can be used to decontaminate the skin and mucosa for the prevention of infections;

2) Vascular disorders: vascular diseases such as the different kinds of macula degeneration in ophthalmology, treatment of atherosclerotic plaques, prevention and/or treatment of vascular (re)stenosis or aneurysms, arteriovenous malformations and other vascular anomalies;

3) Oncology: as an alternative or addition to the standard treatment of tumors and pre-cancerous lesions such as pancreas head cancer, tumors of the brain, lung, cervix, uterus, urinary bladder, bile bladder, stomach, gut, thyroid and oesophagus (including Barret's oesophagus), prostate cancer, head and neck cancers (including cancers of the oral cavity, ears, nose, larynx and pharynx) and kidney tumors;

4)- Ophthalmology disorders: disorders in the eye such as age-related macula degeneration, secondary cataract, infections, immunological diseases and tumors;

5) Gynecological or urological disorders: urogenital diseases such as uterus bleedings, endometriosis, benign prostate hypertrophy and for use in endometrial ablation;

6) Immunological disorders: diseases caused by aberrations of the immune system or increased inflammatory reactions such as multiple sclerosis, rheumatoid arthritis, Inflammatory Bowel Disease (including colitis ulcerosa and Crohn's disease), scleroderma and thyroiditis;

7) Oral cavity or nasopharyngeal disorders, including dentistry applications, for example disorders in the oral cavity such as decontamination of root canals, treatment and/or prevention of gum disease and treatment of wounds or other mucosal disorders.

The porphyrin derivative in a pharmaceutical composition according to the invention may be present in any suitable form, including as its acid or basic addition salt or the free base and free acid thereof and the pharmaceutical composition will generally include a pharmaceutically acceptable carrier or excipient. In general, porphyrins not containing a metal ion will be preferred.

In addition, the derivatives according to the present invention are useful

1) for photodetection of malignant and pre-malignant lesions for instance in the bladder, lung or esophagus;

2) for decontamination or pathogen (such as gram positive and negative bacteria, viruses, parasites, prions and fungi) reduction of liquids such as biological fluids (including donor blood, stem cell containing fluids, bone marrow purging) and contaminated water;

3) for decontamination or pathogen reduction of surfaces either by using liquid photosensitizers or by coupling them directly to the said surface;

4) for use as insecticide.

The appended claims are included in the description by reference.

The invention will now be illustrated with reference to the following examples, and with reference to the drawing, wherein

Scheme S1 depicts the conversion of mesoporphyin dimethylester to the corresponding acrylonitrile derivative via the formyl derivative; (I=nickel (II) acetate tetrahydrate, dimethylformamide, reflux; II=methylformanilide, phosphorus oxychloride, dichloroethane, RT.; III=diethyl phosphono acetonitrile, sodium hydride, tetrahydrofuran, reflux);

Scheme S2 depicts the conversion of the acrylonitrile derivative of scheme 1 to the peri-condensed quinoporphyrin according to the invention (IV=trichloroacetic acid, 175° C., 2 minutes; V=concentrated sulfuric acid, RT.);

Scheme S3 depicts the proposed reaction mechanism of the conversion of the acrylonitrile derivative of scheme 1 to a peri-condensed quinoporphyrin according to the invention using an acid;

Scheme S4a and S4b depict the proposed reaction mechanisms of the conversion of the acrylonitrile derivative of scheme 1 to peri-condensed quinoporphyrins (8 and 9) according to the invention using a Vilsmeier reagent.

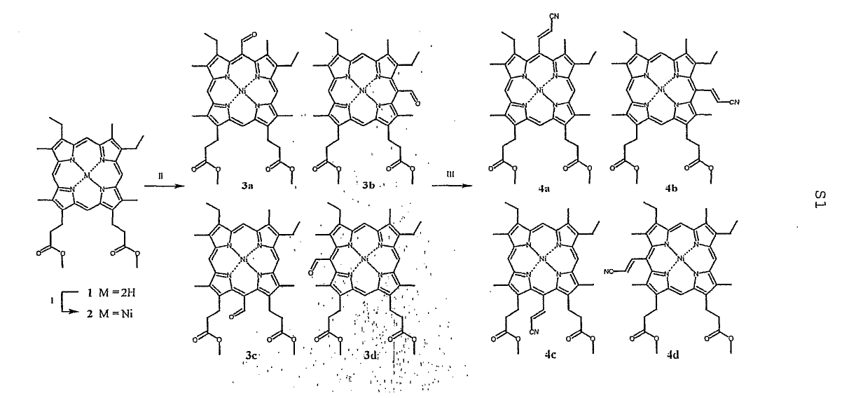

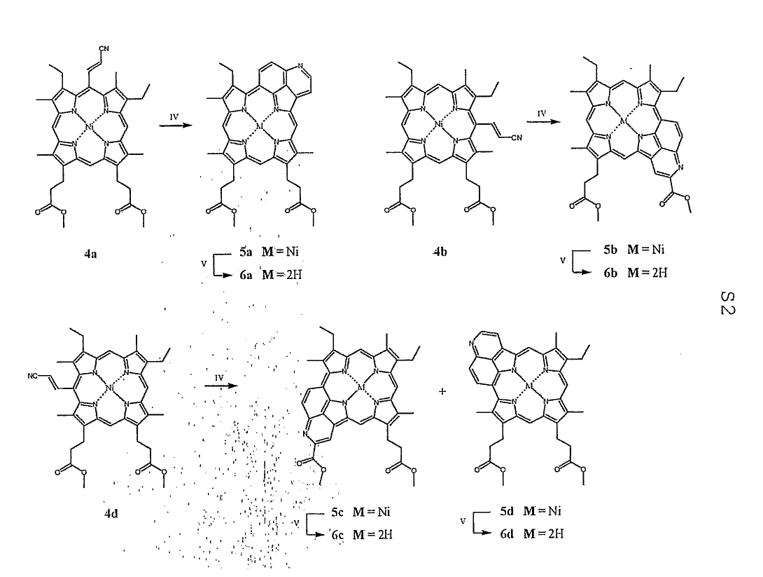

FIG. 1 shows the photodynamic activity of a derivative according to the invention; and

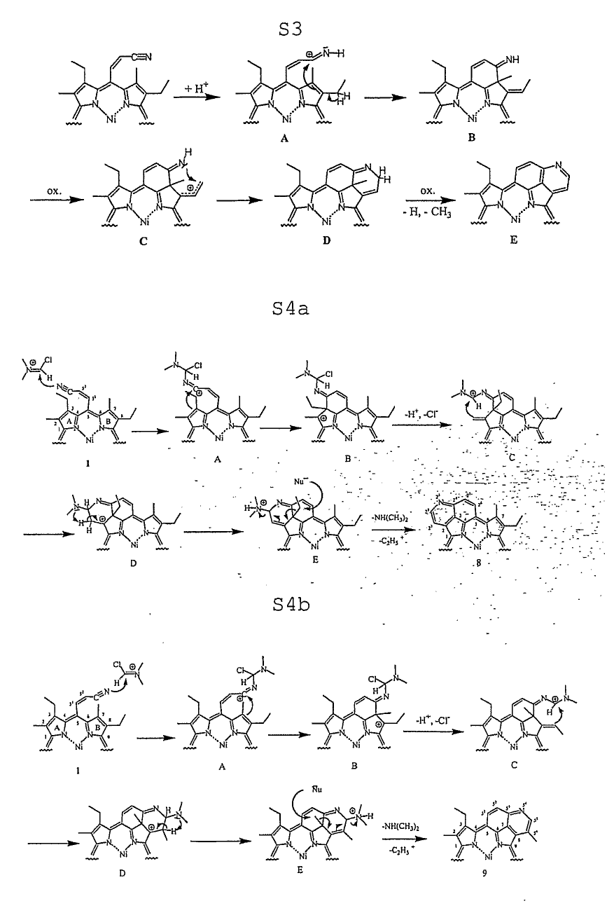

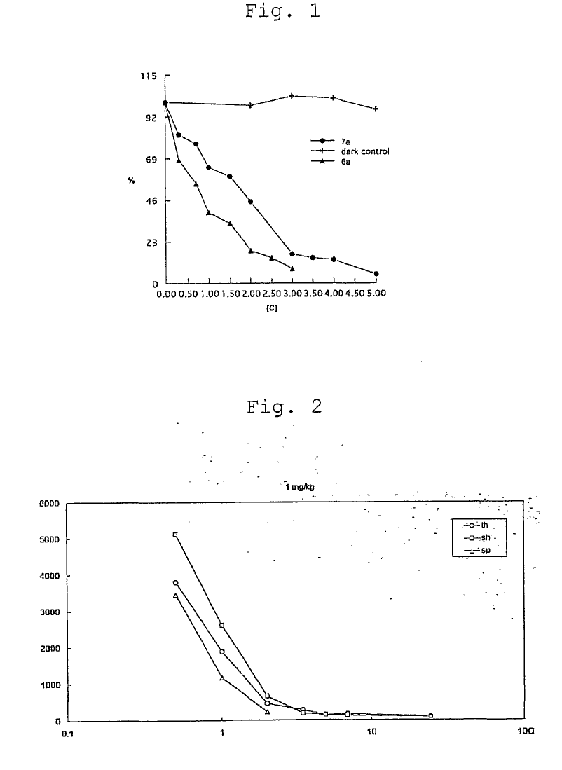

FIG. 2 shows the fast clearance of a porphyrin derivative according to the invention in mice.

### EXAMPLE 1

Step A

Protoporphyrindimethylester (Sato Pharmaceuticals Ltc, Tokyo, Japan) was used for the preparation of mesoporphyrin-dimethylester (1) according to the method disclosed by Fuhrop, J. H. et al (Porphyrins and Metalloporphyrins, supra) 110 g (0,18 mole) of mesoporphyrindimethylester (1), dissolved in 1,5 l of dimethylformamide containing 0,2 mol of nickel (II) acetate, was refluxed for 15 minutes. After evaporation of the solvent under vacuum at 80° C., the subsequent chromatography on silica yielded 103 grams (0,16 mol, 86%) of the mesoporphyrindimethylester-nickel complex 2.

Step B

A Vilsmeier formylation was conducted with methylformanilide and POCl₃ in 1,2-dichloroethane (Vilsmeier, A. et al, Ber. 60, p. 119, (192-7), Minkin et al., Chem. Rev. 74, p. 87-99 (1974)-). This yields a mixture of monoformyl derivatives 3a-3d.

Step C

The mixture of four monoformyl derivatives 3a-3d obtained in Step B was converted into the corresponding meso-acrylonitrile derivatives 4a-4d via a Horner-Emmens reaction (Van den Berg, E. M. M. et al Recl. Chim. Trav. Pays-Bas, 109(3), p. 160-167 (1990), Boutagy et al. Chem. Rev. 74, p. 87-99, (1974)) with the anion of diethylphosphonoacetonitril. The crude mixture was first separated into two fractions by silicagel chromatography using dichloromethane as the eluent. The first fraction (20 g) mainly contained 4a, but also 4b and 4d in a 3:1:2 ratio. The second fraction (57 g) contained all isomers 4a, 4b, 4c and 4d. Fractional crystallisation of the first fraction from a mixture of dichloromethane and hexanes by slow evaporation of dichloromethane yielded 6,6 grams of pure isomer 4a as a bright red solid. The mother liquor contained mainly 4b and 4d and was added to the second fraction. Crystallisation of this mixture gave 63 grams of a mixture of 4b and 4d (ratio 1:2) as a dark green or black crystalline material. The mother liquor contained a mixture of 4a, 4b, 4c and 4d. From NMR it can be seen that in the crude product mixture contained the four isomers 4a, 4b, 4c and 4d in a ratio of 4a: 4b: 4c: 4d=:1:1:0.4:2. This ratio corresponds to what was expected. Introduction of a formyl group at position 20 (3d) experiences the least amount of sterical hindrance because that is where the smallest (methyl) groups are. During the formation of 3a and 3b the sterical hindrance is caused by one methyl group and a larger ethyl group, whereas the formation of 3c involves a more restricting sterical interaction with two-ethyl groups.

Step D (according to the invention)

Refluxing of pure 4a in trichloroacetic acid at 175° C. leads surprising to a new peri-annulated quinoporphyrin-product (5a). The oxidizing agent performing the final oxidation (closing the heterocyclic ring of the quinoline-ring system) to result in the new product is oxygen from the air, similar to what is known for other reactions involving porphyrins (see Woodward, J., Am.. Chem. Soc.. (1960) 38., pp. 3800-3802). The λmax of this new compound 5a is, compared with mesoporphyrindimethylester 1, shifted towards the red (respective values for λmax are: 567 nm and 638 nm).

Treatments of the mixture of 4b and 4d yielded a mixture of 5b (from 4b) and 5c and 5d (from 4d) .

If desired the metal ion used (here nickel) may be removed in a conventional manner (5 min. conc. sulphuric acid at room temperature, the time required to dissolve completely), yielding the corresponding compounds 6a-6d. The full names are shown in table 1. Noteworthy are the maximum absorption wavelengths for 6a, 6b, 6c and 6d which are 681, 688, 688 and 683 nm respectively (in both dichloromethane and methanol),which are very long for entirely unsaturated porphyrin ring systems. Benzylation of the quinoline-ring system nitrogen with benzyl bromide/ potassium iodide leads to formation of a cationic benzylated quinoporphyrin (18 N-benzylquinolinium[4,4a,5,6-efg]-annulated mesoporphyrin dimethylester with an absorption maximum at 755 nm. Hydrolysation of this compound yields N-benzylquinolinium[4,4a,5,6-efg]-annulated mesoporphyrin (19).

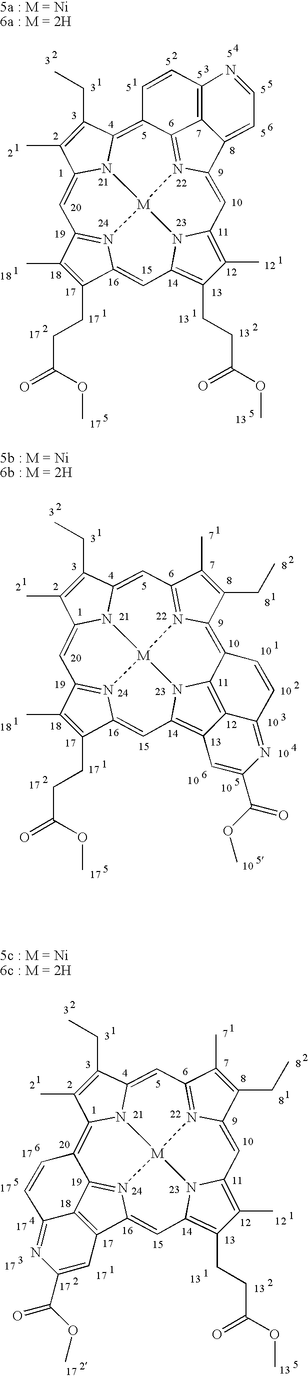

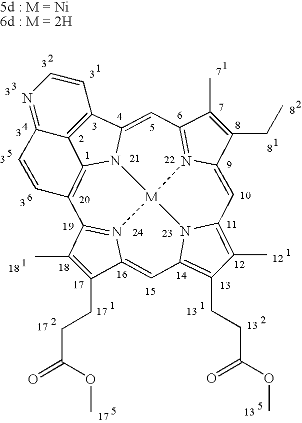

Step E (hydrolysis)

A solution of 0.61 gram of 6a in 25 ml tetrahydrofuran (THF) was mixed with a solution of 0.20 gram sodium hydroxide in 120 ml of water and refluxed during 1 hour. The reaction was followed with silicagel thin layer chromatography using THF as the eluens. When the reaction is finished THF and water were removed by distillation under reduced pressure until the volume was approximately 20 ml. The crude product was purified over 10 grams of CM Sephadex cation exchanger (bead size 40-120 μm) from Sigma-Aldrich, which was first treated with 1 M hydrochloric acid and then washed with demineralized water. First the porphyrin solution was carried onto the sephadex gel, and washed with demineralized water to remove salts. The purified porphyrin was eluted with 1% ammoniumhydroxide in water. The resulting brown solution was evaporated to dryness, then demineralized water was added and again the solution was evaporated to dryness to remove traces of ammonia. This was repeated two times until the porphyrin does not completely dissolve in demineralized water (which is slightly acidic). Yet this solubility of about 0.5 mg/ml at about pH 8 is better than for other porphyrin compounds such as MTHPC which is currently used in photodynamic therapy. 0.45 grams (77%) of the dicarboxylic acid 7a was obtained.

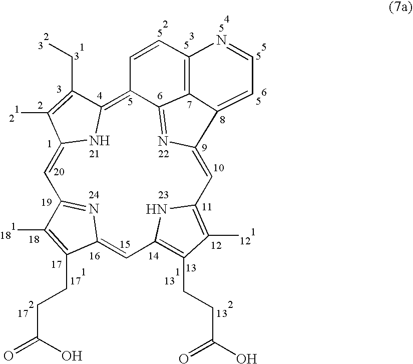

### EXAMPLE 2

### Step F

The product mixture obtained from the Vilsmeier reaction on 400 gram of 2 has been separated into three fractions using silicagel chromatography with dichloromethane as the eluens. The first fraction contained 25 grams of a mixture of 3a (75%) , 3b (8%) and 3.d (17%). Crystallization by slow evaporation of dichloromethane from a mixture of dichloromethane and ethanol yielded 12.1 grams (17.8 mmol) of 5-formylmesoporphyrin dimethylester nickel(II) complex 3a.
This compound was used for the synthesis of 10.
(The second fraction contained 42.0 gram of a mixture of 3a, 3b and 3d in a ratio of 3:1:2 and was used for the synthesis of compounds 4a, 4b, 4c and 4d.)

### Step G

1.02 gram (1.50 mmol) of 3a was dissolved in a mixture of 50 ml dimethylformamide, 50 ml pyridine and 4.0 ml of malononitrile. The mixture was refluxed at 140° C. during 4 hours after which the solvent was evaporated under reduced pressure. The crude 10a 5-(2′,2′-dicyanovinyl)mesoporphyrin dimethylester nickel(II) complex was purified over silicagel using a mixture of 1% of methanol in dichloromethane. This yielded 1.03 gram (94%) of pure 10.

### Step H

Reaction of 950 mg (1.31 mmol) of 10 followed by work-up has proceeded using the procedure described in Step C yielded 96 mg (0.132 mmol, 10%) of the 9′-aminocarbonylquino[4,4a,5,6-efg]-annulated 7-demethyl-8-deethylmesoporphyrin dimethylester nickel(II) complex 11.

The cyclization reaction with the Vilsmeier reagent prepared from dimethylformamide is similar to the cyclization reaction with trichloroacetic acid. The initial step is attack at the nitrogen of the nitrile function, followed by a series of steps that lead to the quinoline formation.

Without wishing to be bound by any particular theory, our experiments to investigate the reaction mechanism made us to believe that the localized positive charge in the Vilsmeier reagent make it a hard electrophile which makes it have a preference for the hard nitrogen atom of the nitrile function. This also takes place when an acid is used for the cyclization reaction.

In the case of the cyclization with the Vilsmeier reagent obtained from dimethylformamide two peri-condensed quinoporphyrins are formed. After attack on the nitrile-nitrogen of the Vilsmeier reagent, the carbenium ion A is formed (scheme 4). Quinoporphyrin 8, which is the main product, is formed via attack of this carbenium ion at carbon 3 of pyrrole-ring A, while the minor product 9 is formed via attack on carbon 7 of pyrrole-ring B.

Here, the carbon atom at position 5 which carries the acrylonitril substituent is designated Cα and the carbon atoms at the positions 2 and 8 are Cδ. When the starting material is 4a, it can be seen that in contrast to the quinoline-ring system formation under acid conditions (Scheme S3) the Vilsmeier reagent now contributes a carbon atom that is incorporated into the quinoline system), leading to the formation of two quinoporphyrins 8 and 9 (i.e. the porphyrin has two Cδ atoms; Schemes 4a and 4b), whereas under Bronsted acid conditions only one quinoporphyrin is formed.

### Step I

Demetallation of 11 (90 mg, mmol) following the procedure as described in Stap D yielded 67 mg (0.10 mmol, 81%) of the free base 12 9′-aminocarbonyl-quino[4,4a,5, 6-efg]-annulated 7-demethyl-8-deethylmesoporphyrin dimethylester.

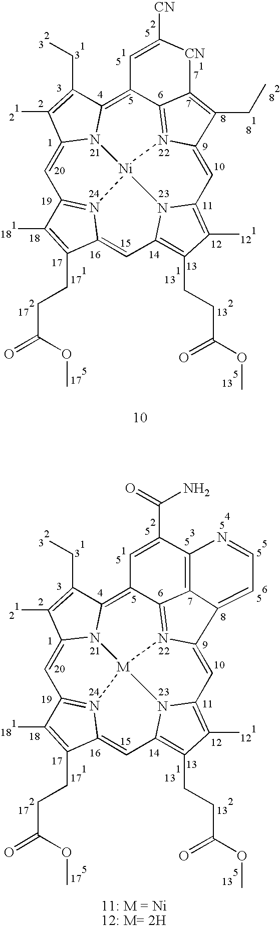

### EXAMPLE 3

Quino [4,4a, 5,6-bcd] -2-demethyl-3-deethylmesoporphyrin dimethylester nickel complex (8) and 3′-methylquino[4,4a,5,6-efg]-7-demethyl-8-deethylmesoporphyrin dimethylester nickel complex (9)
a) Preparation of Vilsmeier reagent
POC1₃ (0.40 ml, 4.3 mmol) was added dropwise to an ice-cold mixture of dimethylformamide (0.55 ml, 4.5 mmol) and 1.0 ml of chloroform, after which the mixture was stirred for 20 minutes at room temperature.
b) The mixture obtained under a) was added to a solution of 500 mg (0.71 mmol) of 5-(2′-cyanovinyl)mesoporphyrin dimethylester nickel complex obtained in Step C of example 1 in 20 ml chloroform. The reaction was followed with TLC using a mixture of 1% methanol in dichloromethane as the eluens. After 16 hours of stirring at room temperature an aqueous solution of sodium acetate was added until the pH was 7 and the reaction mixture was stirred until hydrolysis was complete. Then the chloroform was removed by distillation under reduced pressure, the solid mixture obtained in this way was filtered off and dissolved in dichloromethane. With silicagel flash chromatography a complex mixture of formylated products (350 mg, 0.48 mmol, 67%) was obtained using 2% methanol in dichloromethane as the eluens. When using 5% methanol in dichloromethane a green fraction was collected which contained 45 mg of an impure mixture containing quino-annulated porphyrins as recognized from the v-shaped spot on the TLC obtained with 80% THF and 20′ diethylether (v/v). This latter fraction was purified over silicagel for a second time using a mixture of 60% THF and 40% diethylether (v/v). This yielded 25 mg of a mixture containing 8 and 9 in a 2/1 ratio.

TABLE 1
Overview of most of the compounds mentioned
in this application
(those of the schemes S1-S4 are underlined)

| | |
|---|---|
| | (8) |
| 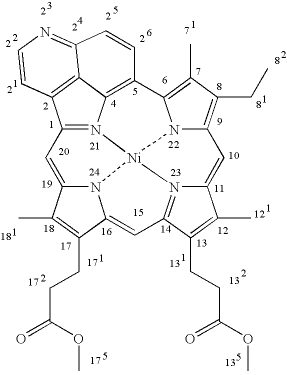 | |
| 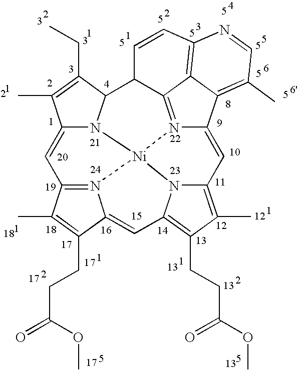 | (9) |

| | |
|---|---|
| 1 | mesoporphyrin dimethylester |
| 2 | mesoporphyrin dimethylester nickel (II) complex |
| 3a | 5-formylmesoporphyrin dimethylester nickel (II) com- |
| | plex |
| 3b | 10-formylmesoporphyrin dimethylester nickel (II) |
| | complex |
| 3c | 15-formylmesoporphyrin dimethylester nickel (II) |
| | complex |
| 3d | 20-formylmesoporphyrin dimethylester nickel (II) |
| | complex |
| 4a | 5-(2′-cyanovinyl)mesoporphyrin dimethylester |
| | nickel (II) complex |
| 4b | 10-(2′-cyanovinyl)mesoporphyrin dimethylester |
| | nickel (II) complex |
| 4c | 15-(2′-cyanovinyl)mesoporphyrin dimethylester |
| | nickel (II) complex |
| 4d | 20-(2′-cyanovinyl)mesoporphyrin dimethylester |
| | nickel (II) complex |
| 5a | quino[4,4a,5,6-efg]-annulated 7-demethyl-8- |
| | deethylmesoporphyrin dimethylester nickel (II) complex |
| 5b | 2′-methoxycarbonylquino[4,4a,5,6-jkl]-annulated 12- |
| | demethyl-13-de[2-(methoxycarbonyl)ethyl]mesoporphyrin dimeth- |
| | ylester nickel (II) complex |
| 5c | 2′-methoxycarbonylquino[4,4a,5,6-qrs]-annulated 18- |
| | demethyl-17-de[2-(methoxycarbonyl)ethyl]mesoporphyrin dimeth- |
| | ylester nickel (II) complex |
| 5d | quino[4,4a,5,6-abt]-annulated 2-demethyl-3- |
| | deethylmesoporphyrin dimethylester nickel (II) complex |
| 6a | quino[4,4a,5,6-efg]-annulated 7-demethyl-8- |
| | deethylmesoporphyrin dimethylester |
| 6b | 2′-methoxycarbonylquino[4,4a,5,6-jkl]-annulated 12- |
| | demethyl-13-de[2-(methoxycarbonyl)ethyl]mesoporphyrin dimeth- |
| | ylester |
| 6c | 2′-methoxycarbonylquino[4,4a,5,6-qrs]-annulated 18- |
| | demethyl-17-de[2-(methoxycarbonyl)ethyl]mesoporphyrin dimeth- |
| | ylester |
| 6d | quino[4,4a,5,6-abt]-annulated 2-demethyl-3- |
| | deethylmesoporphyrin dimethylester |
| 7a | quino[4,4a,5,6-efg]-annulated 7-demethyl-8- |
| | deethylmesoporphyrin |
| 7b | 2′-methoxycarbonylquino[4,4a,5,6-jkl]-annulated 12- |
| | demethyl-13-de[2-(methoxycarbonyl)ethyl]mesoporphyrin |
| 7c | 2′-methoxycarbonylquino[4,4a,5,6-qrs]-annulated 18- |
| | demethyl-17-de[2-(methoxycarbonyl)ethyl]mesoporphyrin |
| 7d | quino[4,4a,5,6-abt]-annulated 2-demethyl-3- |
| | deethylmesoporphyrin |
| 8 | quino[4,4a,5,6-bcd]-2-demethyl-3-deethyl- |
| | mesoporphyrin dimethylester nickel (II) complex |
| 9 | 3′-methylquino[4,4a,5,6-efg]-7-demethyl-8- |
| | deethylmesoporphyrin dimethylester nickel (II) complex |
| 10 | 5-(2′,2′-dicyanovinyl)mesoporphyrin dimethylester |
| | nickel (II) complex |
| 11 | 9′-aminocarbonylquino[4,4a,5,6-efg]-7-demethyl-8- |
| | deethylquinoporphyrin dimethylester nickel (II) complex |
| 12 | 9′-aminocarbonylquino[4,4a,5,6-efg]-7-demethyl-8- |
| | deethylquinoporphyrin dimethylester |
| 13 | 9′-aminocarbonylquino[4,4a,5,6-efg]-7-demethyl-8- |
| | deethylquinoporphyrin |
| 14 | quino[4,4a,5,6-bcd]-2-demethyl-3-deethyl- |
| | mesoporphyrin dimethylester (derived from 8) |
| 15 | quino[4,4a,5,6-bcd]-2-demethyl-3-deethyl- |
| | mesoporphyrin |
| 16 | 3′-methylquino[4,4a,5,6-efg]-7-demethyl-8- |
| | deethylmesoporphyrin dimethylester (derived from 9) |
| 17 | 3′-methylquino[4,4a,5,6-efg]-7-demethyl-8- |
| | deethylmesoporphyrin |
| 18 | N-benzylquinolinium[4,4a,5,6-efg]-annulated |
| | mesoporphyrin dimethylester |
| 19 | N-benzylquinolinium[4,4a,5,6-efg]-annulated |
| | mesoporphyrin |

TABLE 2
Overview of mass spectrometry data (HR-FAB MS
[M + H]) of some compounds from the examples

| compound | Brutoformula | m/z (found) | m/z (calcd) |
|---|---|---|---|
| 2 | ¹²C₃₆¹H₄₁¹⁴N₄¹⁶O₄⁵⁸Ni⁺ | 651.2469 | 651.2481 |
| 3a-d | ¹²C₃₇¹H₄₁¹⁴N₄¹⁶O₅⁵⁸Ni⁺ | 679.2414 | 679.2430 |
| 4a | ¹²C₃₉¹H₄₂¹⁴N₅¹⁶O₄⁵⁸Ni⁺ | 702.2593 | 702.2590 |
| 4b + 4d | ¹²C₃₉¹H₄₂¹⁴N₅¹⁶O₄⁵⁸Ni⁺ | 702.2585 | 702.2590 |
| 4c | ¹²C₃₉¹H₄₂¹⁴N₅¹⁶O₄⁵⁸Ni⁺ | 702.2618 | 702.2590 |
| 5a | ¹²C₃₈¹H₃₆¹⁴N₅¹⁶O₄⁵⁸Ni⁺ | 684.2108 | 684.2121 |
| 5c | ¹²C₃₈¹H₃₆¹⁴N₅¹⁶O₄⁵⁸Ni⁺ | 684.2112 | 684.2121 |
| 5d | ¹²C₃₈¹H₃₆¹⁴N₅¹⁶O₄⁵⁸Ni⁺ | 684.2149 | 684.2121 |
| 6a | ¹²C₃₆¹H₃₈¹⁴N₅¹⁶O₄⁺ | 628.2921 | 628.2924 |
| 6b | ¹²C₃₆¹H₃₈¹⁴N₅¹⁶O₄⁺ | 628.2912 | 628.2924 |
| 6c | ¹²C₃₆¹H₃₈¹⁴N₅¹⁶O₄⁺ | 628.2933 | 628.2924 |
| 6d | ¹²C₃₆¹H₃₈¹⁴N₅¹⁶O₄⁺ | 628.2869 | 628.2924 |
| 8 | ¹²C₃₈¹H₃₆¹⁴N₅¹⁶O₄⁵⁸Ni⁺ | 684.2134 | 684.2121 |
| 9 | ¹²C₃₉¹H₃₈¹⁴N₅¹⁶O₄⁵⁸Ni⁺ | 698.2283 | 698.2277 |
| 10 | ¹²C₄₀¹H₄₁¹⁴N₆¹⁶O₄⁵⁸Ni⁺ | 727.2548 | 727.2543 |

Biological Experiments

### EXAMPLE 4

FIG. 1 depicts the cell survival of Chinese hamster ovary cells as measured with the standard MTT survival assay (Carmichael, J., et al (1987), Cancer Research, 47, pp. 936-942). On the y-axis is plotted the percentage cell survival as compared to a non-treated control, on the x-axis the concentration of the sensitizers 6a and 7a according to the invention in micrograms per ml incubation medium. The cultured cells were incubated with medium containing the sensitizer for 4 hours. Thereafter the medium was replaced by phosphate buffer and the cells were illuminated using broad band white light (30 mW/cm²) for 15 minutes. Subsequently the buffer was replaced by fresh culture medium and the survival was assessed 24 hours after the illumination.

### EXAMPLE 5

To determine the acute toxicity, female CBA/CA mice were injected intravenously over a period of 5 to 7 minutes with 10 mg/kg of bodyweight of a 1 mg/ml solution of 7a in polyethyleneglycol 400: ethanol water=30:20:50 (by volume). All mice survived the injection procedure and the period thereafter. There was no difference in behaviour or appearance of the mice that received 7a (n=3) or vehicle only (n=3). In addition, after pathological inspection, there was no difference in the macroscopic appearance of the major organs in thorax and abdomen between the experimental and control mice.

### EXAMPLE 6

Photodynamic therapy of mammals is impeded by the inadequate properties of the porphyrins available in the art. Patients treated must remain out of daylight for days after the treatment because of the long half-life of porphyrins in the body. Cases of severe damage to the blood vessels in which porphyrins were injected are known, and this is a serious drawback to what could be a very important technique thanks to the localized treatment (by illuminating the sites to be treated) Hairless mice were injected with compound 7a according to the invention at 1 mg/ml. Clearance of this porphyrin derivative was measured using the intrinsic fluorescence of the porphyrin. Using fiber optics (Y-shaped fiber), parts of the body (thigh (th), to measure fluorescence in muscle, shoulder (sh) to measure bone, ear to measure fluorescence in the skin and liver and spleen (sp) to measure the fluorescence in these organs) were illuminated with light (408 nm; through one branch of the Y-fiber) to excite the Soret-band (note: with photodynamic therapy absorption bands more to the red are excited), and fluorescence at 470 nm was measured (fluorescence captured in the base of the Y-shaped fiber was guided to a photo-detector via the second branch) . Decay in the measured fluorescence and the organ distribution strongly suggest that 7a is behaves as a compound that does not leave the blood vessel.

FIG. 2 shows the fast clearance of the porphyrins according to the invention (Y-axis: fluorescence in arbitrary units; X-axis time in hours), promising to overcome the major disadvantage of Foscan® (mTHPC), which also has to be used at much lower concentration to avoid acute toxicity. The urine of the mice was coloured, also indicating rapid clearance from the blood stream.

1. A method of preparing a porphyrin derivative starting from a meso-substituted porphyrin compound, characterized in that a meso-(2′-cyanovinyl)-substituted porphyrin compound of which the vinyl is optionally substituted is used as the meso-substituted porphyrin compound, wherein said meso-(2′-cyanovinyl)-substituted porphyrin compound, in a form in which its porphyrin macrocycle is complexed with a bivalent metal ion
i) is subjected to
an acid for which 0<pKa<5
and
an oxidizing agent,
with the restriction that if the carbon atom of the porphyrin macrocycle at which the (2′-cyanovinyl) substituent is attached is designated Cα, there must be a substituent attached to Cδ, counting along the perimeter of the porphyrin macrocycle, said substituent comprising a —C—C motif directly attached at the Cδ carbon atom;
or
ii) is subjected under aprotic conditions to a Vilsmeier reagent having a reactive motif 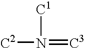
containing a quaternary nitrogen atom which is directly linked to two carbon atoms C¹, C² wherein said carbon atoms are not part of a unsaturated or aromatic moiety, and which quaternary nitrogen atom is directly linked to a carbon atom C³ via a double bond, said carbon atom C³ carrying a halogen atom chosen from fluoro, chloro, bromo and iodo
with the restriction that if the carbon atom of the porphyrin macrocycle at which the (2′-cyanovinyl) substituent is attached is designated Cα, there must be a substituent attached to Cδ, counting along the perimeter of the porphyrin macrocycle, said substituent comprising a —CH motif directly attached at the Cδ carbon atom;
to convert said meso-(2′-cyanovinyl)-substituted porphyrin compound into a porphyrin derivative having a quinoline-ring system peri-condensed to the porphyrin ring, and optionally the bivalent metal ion is removed or replaced by another metal ion, and optionally the nitrogen atom of the quinoline-ring system ring is quaternized.

2. The method according to claim 1, characterized in that for alternative step i) a meso-(2′-cyanovinyl)-substituted porphyrin compound of formula (I) is used as the starting compound, 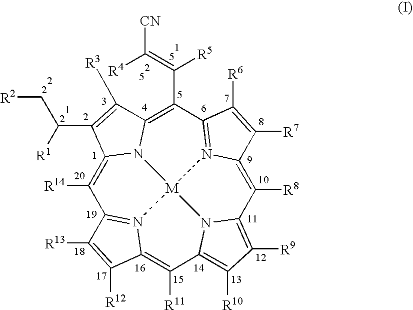
or wherein for alternative step ii) meso-(2′-cyanovinyl)-substituted porphyrin compound of formula (III) is used as the starting compound 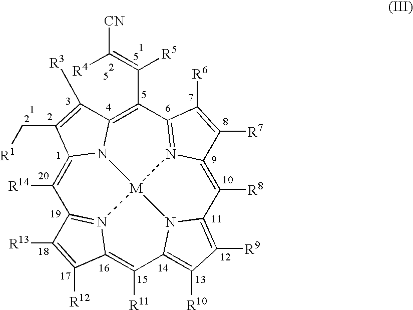
wherein
R¹, R² represent independently of each other hydrogen, linear or branched (C₁₋₈) alkyl, or linear or branched (C₁₋₈)alkyl C(O)O (C₁₋₈)alkyl, wherein the groups comprising alkyl may optionally be substituted with fluoro, chloro, bromo, iodo, nitrile, (C₁₋₈) thioether, and (C₁₋₈) alkoxy;
R³ represents H or (C₁₋₈) alkyl;
R⁴ and R⁵, represent, independently of each other, hydrogen, nitrile, monocyclic, bicyclic or tricyclic (C₆₋₁₄) aryl, or (C₁₋₄) alkyl wherein the aryl and alkyl group may optionally be substituted with fluoro, chloro, bromo, iodo, nitrile, (C₁₋₈) thioether, and (C₁₋₈) alkoxy;
R⁶ to R¹⁴ represent independently of each other, hydrogen, linear or branched (C₁₋₈) alkyl, linear or branched (C₁₋₈)alkyl C(O)O (C₁₋₈)alkyl, wherein n is an integer of 0 to 4, CH₂═CH—, a monocyclic, bicyclic or tricyclic (C₃-C₁₄) aryl, which aryl may optionally contain one or more nitrogen atoms as heteroatoms; and R⁸, R¹¹, and R¹⁴ may in addition represent an acrylonitrile group substituted with R^{4′} and R⁵′, wherein R^{4′′ and R}⁵′ are as defined for R⁴ and R⁵;
and
M represents a bivalent metal ion,
wherein the compound of formula (I) or (III) is converted into the corresponding porphyrin derivative of formula (II) comprising a quinoline-ring system fused to the porphyrin ring 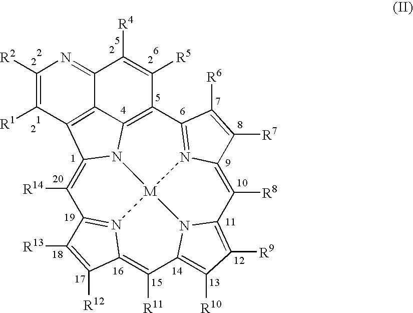
wherein the substituents have the meanings given above, and depending on the meaning of R⁸, R¹¹, and R¹⁴ and the correspondence of an adjacent R⁷, R⁹, R¹⁰, R¹², and R¹³ with R³ optionally more than one quinoline-ring system peri-condensed to the porphyrin ring is present.

3. The method according to claim 1, characterized in that the nitrogen atom of the peri-condensed quinoline-ring system ring in formula (II) is quaternized.

4. The method according to claim 1, characterized in that the meso-(2′-cyanovinyl)-substituted porphyrin compound is prepared by introducing a formyl or acetyl residue at a meso position of a porphyrin compound, whereafter the mesoformylporphyrin thus formed is converted into the meso-(2′-cyanovinyl) derivative.

5. The method according to claim 4, characterized in that the mesoformylporphyrin formed is converted into the meso-(2′-cyanovinyl)-substituted porphyrin compound by reaction with diethylphosphonoacetonitril.

6. The method according to claim 1, characterized in that the porphyrin starting compound for the preparation of the meso-(2′-cyanovinyl) porphyrin is chosen from the group of i) hemin, and ii) heme.

7. The method according to claim 1, characterized in that Ni²⁺ is used as the bivalent metal ion.

8. The method according to claim 1, characterized in that a Brönsted-acid is used with the provisio that 0<pKa<5, the reaction being carried out at a temperature above 140° C.

9. The method according to claim 1, characterized in that the Vilsmeier reagent used is of the formula (IV) 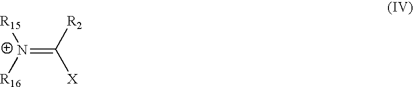
wherein
R15 and R16 are, independently of each other, linear or branched C₁₋₈ alkyl,
X is fluoro, chloro, bromo and iodo, and
R2 is hydrogen, linear or branched (C₁₋₈) alkyl, or linear or branched (C₁₋₈)alkyl C(O)O (C₁₋₈)alkyl, wherein the groups comprising alkyl may optionally be substituted with fluoro, chloro, bromo, iodo, nitrile, (C₁₋₈) thioether, and (C₁₋₈) alkoxy.

10. The method according to claim 9, characterized in that X is chloro or bromo.

11. Porphyrin derivatives, wherein said derivatives are:
2′-methoxycarbonylquino[4,4a,5,6-jkl]-annulated 12-demethyl-13-de[2-(methoxycarbonyl)ethyl]mesoporphyrin dimethylester;
2′-methoxycarbonylquino[4,4a,5,6-qrs]-annulated 18-demethyl-17-de[2-(methoxycarbonyl)ethyl]mesoporphyrin dimethylester;
quino[4,4a,5,6-abt]-annulated 2-demethyl-3-deethylmesoporphyrin dimethylester;
quino[4,4a,5,6-efg]-annulated 7-demethyl-8-deethylmesoporphyrin;
2′-methoxycarbonylquino[4,4a,5,6-jkl]-annulated 1 2-demethyl-13-de[2-(methoxycarbonyl)ethyl]mesoporphyrin;
2′-methoxycarbonylquino[4,4a,5,6-qrs]-annulated 1 8-demethyl-17-de[2-(methoxycarbonyl)ethyl]mesoporphyrin;
quino[4,4a,5,6-abt]-annulated 2-demethyl-3-deethylmesoporphyrin;
quino[4,4a,5,6-bcd]-2-demethyl-3-deethyl-mesoporphyrin dimethylester;
quino[4,4a,5,6-bcd]-2-demethyl-3-deethyl-mesoporphyrin;
3′-methylquino[4,4a,5,6-efg]-7-demethyl-8-deethylmesoporphyrin dimethylester;
3′-methylquino[4,4a,5,6-efg]-7-demethyl-8-deethylmesoporphyrin;
9′-aminocarbonylquino[4,4a,5,6-efg]-7-demethyl-8-deethylquinoporphyrin dimethylester;
9′-aminocarbonylquino[4,4a,5,6-efg]-7-demethyl-8-deethylquinoporphyrin
N-benzylquinolinium[4,4a,5,6-efg]-annulated mesoporphyrin dimethylester
N-benzylquinolinium[4,4a,5,6-efg]-annulated mesoporphyrin.

12. A porhyrin derivative having a quinoline-ring system peri-condensed to the porphyrin ring.

13. Use of a porphyrin derivative according to claim 12 for the preparation of a pharmaceutical composition of a porphyrin derivative according to the invention for prevention of and/or treating
1) benign, malignant, inflamed and infectious skin and mucosa disorders: skin/mucosa disorders;
2) vascular disorders;
3) tumors and pre-cancerous lesions;
4) ophthalmology disorders;
5) gynecological or urological disorders;
6) immunological disorders;
7) oral cavity or nasopharyngeal disorders.

14. Use of a porphyrin derivative according to claim 12 for the preparation of a composition of a porphyrin derivative according to the invention for the preparation of a composition
1) for photodetection of malignant and pre-malignant lesions;
2) for decontamination or pathogen reduction of liquids such biological fluids and contaminated water;
3) for decontamination or pathogen reduction of surfaces;
4) for use as insecticide.

15. Pharmaceutical composition comprising a porphyrin derivative according to claim 12 together with a pharmaceutically acceptable carrier or excipient.
